(19) United States
(12) Plant Patent Application Publication
(10) Pub. No.: US 2002/0152513 P1
(43) Pub. Date: Oct. 17, 2002

(54) Miniature rose variety 'POULgo003'

(76) Inventors: L. Pernille Olesen, Fredensborg (DK); Mogens N. Olesen, Fredensborg (DK)

Correspondence Address: Mogens N. Olesen, Poulsen Roser Pacific, Inc., 2550 Old Military Road, Central Point, OR, 97502, US

(21) Appl. No.: 09/776,155

(22) Filed: Feb. 2, 2001

(51) Int. Cl. A01H 5/00

(52) U.S. Cl. PLT/121

(57) ABSTRACT

A new miniature rose plant which has abundant, coral-pink flowers and attractive foliage. This new and distinct variety has shown to be uniform and stable in the resulting generations from asexual propagation.

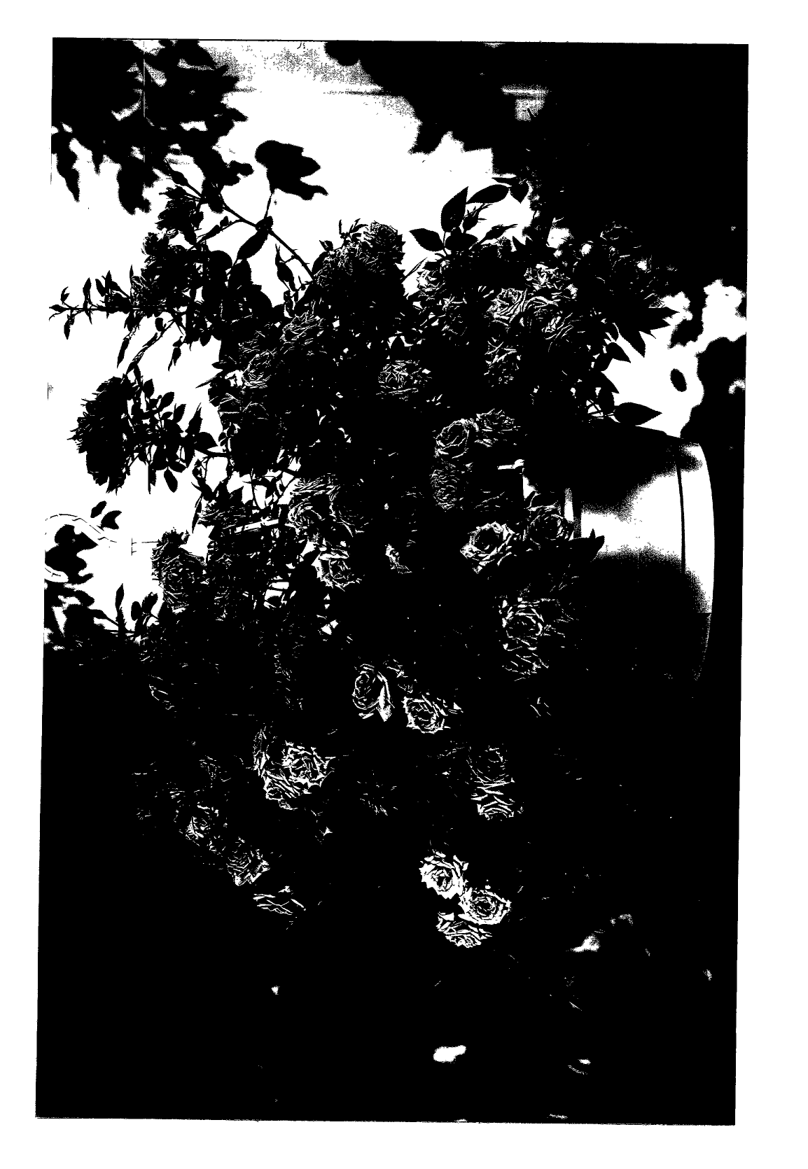

### SUMMARY OF THE INVENTION

The present invention constitutes a new and distinct variety of miniature rose plant which originated from a controlled crossing between an unnamed and unpatented variety, and ‘POULrijk’, described and illustrated in U.S. Plant Patent application Ser. No. 09/261,445, dated Mar. 3, 1999. The two parents were crossed during the summer of 1994 and the resulting seeds were planted in a controlled environment in Fredensborg, Denmark. The new variety is named ‘POULgo003’.

The new variety may be distinguished from its seed parent, an unnamed seedling, by the following combination of characteristics:

1. ‘POULgo003’ has coral-pink flowers, whereas the seed parent has pink flowers.

2. ‘POULgo003’ exhibits pendulant to procumbent growth, while the seed parent has compact, upright growth.

The new rose may be distinguished from its pollen parent, ‘POULrijk’, by the following combination of characteristics:

1. ‘POULrijk’ is a ground cover shrub rose, whereas ‘POULgo003’ is a miniature rose.

2. ‘POULrijk’ has a spreading habit while ‘POULgo003’ exhibits a procumbent habit.

3. ‘POULrijk’ has semi-double, pure pink flowers, while ‘POULgo003’ has double, coral-pink flowers.

The objective of the hybridization of this rose variety was to create a new and distinct variety for garden use with unique qualities, such as:

1. Uniform and abundant flowers;

2. Vigorous, but compact growth when propagated by cuttings;

3. Disease resistance;

4. A miniature rose suitable for use in hanging basket containers in the nursery industry.

This combination of qualities is not present in previously available commercial cultivars of this type and distinguish ‘POULgo003’ from all other varieties of which we are aware.

As part of their rose development program, L. Pernille Olesen and Mogens N. Olesen germinated the seeds from the aforementioned hybridization during winter of 1994 and conducted evaluations on the resulting seedlings in a controlled environment in Fredensborg, Denmark.

‘POULgo003’ was selected in the spring of 1995 by the inventors as a single plant from the progeny of the aforementioned hybridization.

Asexual reproduction of ‘POULgo003’ by traditional budding and rooted cuttings was first done by L. Pernille and Mogens N. Olesen in their nursery in Fredensborg, Denmark in July 1995. This initial and other subsequent asexual propagations conducted in controlled environments have demonstrated that the characteristics of ‘POULgo003’ are true to type and are transmitted from one generation to the next.

### BRIEF DESCRIPTION OF THE DRAWING

The accompanying color illustration shows as true as is reasonably possible to obtain in color photographs of this type the variety rose ‘POULgo003’. Specifically illustrated in SHEET 1 is a plant of ‘POULgo003’ growing in a 20-cm hanging basket.

### DETAILED DESCRIPTION OF THE VARIETY

The following is a description of ‘POULgo003’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULria’, a rose variety from the same inventors described and illustrated in U.S. Plant Patent application Ser. No. 09/274,686 and dated Mar. 24, 1999 are compared to ‘POULgo003’ in Chart 1.

CHART 1

| | ‘POULgo003’ | ‘POULria’ |
|---|---|---|
| Color of open | Red-Purple Group | Red Group 53A to |
| flower, outer | 58C | Red-Purple Group |
| side, middle | | 59C |
| zone. | | |
| Petalage | Double 25+ | Very double, 40-50 |
| Average Bloom | Small, 50-80 mm | Small, 35-40 mm |
| Diameter | | |

Parents:

Seed parent.—Unnamed seedling.

Pollen parent.—‘POULrijk’.

Classification:

Botanical.—Rosa hybrida.

Commercial.—Miniature.

### FLOWER AND FLOWER BUD

Blooming Habit: Continuous.

Flower Bud:

Bud form.—Ovate.

Sepals.—Weak extensions.

Receptacle.—Size: Small.

Peduncle.—Surface: Many hairs and prickles.

Borne.—Many flowering shoots.

Flower Bloom:

Fragrance.—Very weak.

Size.—Small.

Form.—Shape of flower when viewed from the side: Upper part: Flattened convex. Lower part: Flattened convex. Viewed from above: Star-shaped.

Petalage.—Double.

Color:

Petals.—Inner Side: Middle zone: Red Group 52C. Marginal zone: Red Group 52C. Basal petal spot: White Group 155B. Outer side: Middle zone: Red-Purple Group 58C. Marginal zone: Red-Purple Group 58C. Basal petal spot: White Group 155B.

General Tonality: On open flower, Red Group 52C to Red-Purple Group 58C.

Petals:

Petal reflex.—Strong.

Undulation of margin.—Weak.

Size.—Small.

Reproductive Organs:

Filaments.—Color: Yellow.

Hips.—Shape: Pitcher-shaped.

### PLANT

Plant Growth: Spreading. Procumbent to arching. Height: 40 to 60 cm.

Stems:

Thorns.—Incidence: Few short prickles and many long prickles. Shape: Concave.

Plant Foliage:

Leaf size.—Small.

Glossy.—Weak glossiness.

Color.—Upper Leaf Surface: Medium green. Juvenile foliage: Exhibits anthocyanin on young shoots. Anthocyanin intonation: Color: Bronze to Reddish brown.

Plant Leaflets:

Leaflet.—Cross Section: Flat. Margin Undulation: Weak.

Terminal leaflet.—Length of blade: Very short. Width of blade: Very narrow. Shape: Rounded.

Disease Resistance: Above average resistance to mildew, rust, black spot, and Botrytis under normal growing conditions in Hannover, Germany and Fredensborg, Denmark.

Cold Hardiness: The variety ‘POULgo003’ has been found to be cold hardy in Fredensborg, Denmark and Hannover, Germany.

1. A new and distinct variety of rose plant of the miniature rose class, substantially as herein illustrated and described as a distinct and novel rose variety due to its abundant coral-pink flowers, procumbent to arching habit, disease resistance, and extended period of bloom.
